(19) United States
(12) Patent Application Publication
(10) Pub. No.: US 2024/0016860 A1
(43) Pub. Date: Jan. 18, 2024

(54) BACILLUS COAGULANS FOR IMPROVING CONSTIPATION AND APPLICATION THEREOF

(71) Applicant: Thankcome Biological Science and Technology (Suzhou) Co., Ltd., Suzhou, Jiangsu (CN)

(72) Inventors: Xueping YU, Suzhou (CN); Xin MA, Suzhou (CN); Yang YU, Suzhou (CN)

(21) Appl. No.: 18/460,124

(22) Filed: Sep. 1, 2023

Related U.S. Application Data
(63) Continuation of application No. PCT/CN2022/143703, filed on Jan. 16, 2023.

(30) Foreign Application Priority Data
Mar. 14, 2022 (CN) 202210245396.0

(51) Int. Cl. A61K 35/742; A61P 1/10

(52) U.S. Cl. CPC A61K 35/742; A61P 1/10

(57) ABSTRACT

Disclosed are a Bacillus coagulans and its application for improving constipation, belonging to the field of microbial strains. The Bacillus coagulans disclosed in the present application is preserved in China General Microbiological Culture Collection Center (CGMCC) on Nov. 10, 2021, with a preservation number of CGMCC No. 23766 and a preservation address of the Institute of Microbiology, Chinese Academy of Sciences, No. 3, Yard No. 1 Beichen West Road, Chaoyang District, Beijing.

### CROSS-REFERENCE TO RELATED APPLICATIONS

This application is a continuation of PCT/CN2022/143703, filed Jan. 16, 2023 and claims priority of Chinese Patent Application No. 202210245396.0, filed on Mar. 14, 2022, the entire contents of which are incorporated herein by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: sequence_347_033_4962
Creation date: Aug. 14, 2023
Byte size: 2,571 bytes

### TECHNICAL FIELD

The present application relates to the technical field of microbial strains, and in particular to a Bacillus coagulans for improving constipation and an application thereof.

### BACKGROUND

As one of the common symptoms of the digestive system, constipation greatly impairs the health of people. It causes the accumulation of toxins in the body, reduces the function of the organs and may even lead to the occurrence of other diseases, and chronic accumulation of toxins leads to abnormalities in the function of the endocrine system, hormone metabolism imbalance, resulting in an increased burden on the liver. Therefore, it is important to know how to effectively control constipation and the various diseases and abnormalities in the function of the endocrine system that it causes.

The constipation is usually a result of unhealthy lifestyles (e.g. poor diet, bowel habits and lack of exercise), psychological factors, and some digestive disorders. In clinical practice, laxatives and prokinetic agents (5-HT modulators, gastrin agonists and chloride channel activators) are often used to treat constipation; such a measure is effective in improving constipation but are not advisable for long-term administration since prolonged administration of the drugs often leads to side effects such as drug dependence, abdominal pain, diarrhea, nausea, vomiting and headache, which seriously affect the patient's quality of life.

Clinical studies have shown that there is a serious dysbiosis in the intestinal tract of constipated patients and that the treatment of constipation can be achieved by regulating the local micro-ecological environment through the administration of probiotics, which is a new idea in this field of research.

### SUMMARY

The present application provides a Bacillus coagulans for improving constipation and application thereof to solve the above-mentioned problems of the prior art, the Bacillus coagulans is a potential probiotic strain with capacity of improving constipation.

In order to achieve the above objectives, the present application provides the following technical schemes:

the present application provides a Bacillus coagulans, and the Bacillus coagulans has been preserved in the China General Microbiological Culture Collection Center (CGMCC) on Nov. 10, 2021, with a preservation number of CGMCC No. 23766, and a preservation address of the Institute of Microbiology, Chinese Academy of Sciences, No. 3, Yard No. 1, Beichen West Road, Chaoyang District, Beijing. The place of acquisition of this deposited biological material was Suzhou, Jiangsu Providence, China.

The present application also provides an application of the Bacillus coagulans in preparing products for preventing and/or treating constipation.

Optionally, the constipation refers to constipation caused by loperamide hydrochloride.

The present application also provides an application of the Bacillus coagulans in preparing products for ameliorating weight increase caused by loperamide hydrochloride.

The present application also provides a product with an efficacy of improving constipation, with the Bacillus coagulans or secondary metabolites of the Bacillus coagulans as a main active ingredient.

The present application achieves the following technical effects:

the present application discloses the effect of Bacillus coagulans on alleviating the symptoms of drug-induced functional constipation and provides a preliminary discussion of the action mechanism of Bacillus coagulans; the ameliorating effect of this lactobacillus on constipation is analyzed visually by observing the changes in body weight, fecal water content, small intestinal propulsion rate and pathological tissue sections in a loperamide hydrochloride induced functional constipation model of mice; and the effectiveness of the lactobacillus on constipation is then evaluated by serum indicators, mRNA and protein expression levels; the experimental results of the present application provide not only an experimental and theoretical basis for the development of Bacillus coagulans as a health food and pharmaceutical product, but also offer theoretical support for the effectiveness of probiotics in relieving bowel movement; and

the effectiveness of Bacillus coagulans in relieving the symptoms of constipation or restoring the bowel movement of mice has been demonstrated through the experiments of the present application; Bacillus coagulans offers good laxative effects by promoting small intestinal propulsion and increasing fecal water content in mice, as well as regulatory effect on biomarkers related to gastrointestinal motility, thereby regulating intestinal function in constipated mice and alleviating intestinal inflammation; it is demonstrated that Bacillus coagulans is an effective candidate probiotic to reduce the adverse effects of constipation.

### BRIEF DESCRIPTION OF THE DRAWINGS

For a clearer illustration of the technical schemes in the embodiments of the present application or in the prior art, a brief description of the accompanying drawings to be used in the embodiments is given below. It is obvious that the accompanying drawings in the following description are only some embodiments of the present application and that other accompanying drawings are also available to those of ordinary skill in the art without any creative effort.

FIG. 1 shows weight changes of mice during an experiment, in which NC is a group of normal control, GM represents a constipation model group, Bis represents a bisacodyl drug control group, BC-H represents a BC high-dose group, and BC-L represents a BC low-dose group.

FIG. 2 shows changes of fecal water loss rate of mice during the experiment, in which NC is the group of normal control, GM represents the constipation model group, Bis represents the bisacodyl drug control group, BC-H represents the BC high-dose group, and BC-L represents the BC low-dose group.

FIG. 3 shows anatomical diagrams of small intestine, in which NC is the group of normal control, GM represents the constipation model group, Bis represents the bisacodyl drug control group, BC-H represents the BC high-dose group, and BC-L represents the BC low-dose group.

FIG. 4 shows pathological sections (stained by hematoxylin eosin) of small intestine, in which NC is the group of normal control, GM represents the constipation model group, Bis represents the bisacodyl drug control group, BC-H represents the BC high-dose group, and BC-L represents the BC low-dose group.

### DETAILED DESCRIPTION OF THE EMBODIMENTS

Various exemplary embodiments of the present application are now described in detail and this detailed description should not be considered as limiting the present application, but should be understood as a more detailed description of certain aspects, features and embodiments of the present application.

It should be understood that the terms described in the present application are intended to describe particular embodiments only and are not intended to limit the present application. Furthermore, with respect to the range of values in the present application, it is to be understood that each intermediate value between the upper and lower limits of the range is also specifically disclosed. Each smaller range between any stated value or intermediate value within a stated range and any other stated value or intermediate value within a stated range is also included in the present application. The upper and lower limits of these smaller ranges may be independently included or excluded from the scope.

Unless otherwise stated, all technical and scientific terms used herein have the same meaning as is commonly understood by those of ordinary skill in the field described in the present application. Although the present application describes only preferred methods and materials, any methods and materials similar or equivalent to those described herein may also be used in the implementation or testing of the present application. All literature referred to in this specification is incorporated by reference for the purpose of disclosing and describing the methods and/or materials associated with said literature. In the event of conflict with any incorporated literature, the contents of this specification shall prevail.

Without departing from the scope or spirit of the present application, various improvements and variations may be made to specific embodiments of the specification of the present application, as will be apparent to those skilled in the art. Other embodiments derived from the specification of the present application will be obvious to the skilled person. The specification and embodiments of the present application are only exemplary.

As used herein, the terms “comprise”, “include”, “have”, “contain”, etc. are open-ended, i.e. meaning including but not limited to.

### Embodiment 1 Isolation and Identification of Bacillus coagulans

Media: DeMan, Rogosa and Sharpe (MRS) liquid and solid media (Qingdao Hopebio), Murashige & Skoog (MC) liquid and solid medium (Basebio) and brain heart infusion (BHI) liquid and solid media (Qingdao Hopebio).

Salted beans of 10 grams (g) are added to a triangular flask, and after shaking on a shaker for 30 minutes (min), 1 milliliter (mL) is aspirated to a dilution solution of 9 mL to obtain a 10-1 dilution solution, which is sequentially diluted in a gradient for 6 times, and then coated and inoculated into MRS/MC/BHI agar medium in turn, followed by anaerobic inversion incubation at 37 degrees Celsius (° C.) for 72 hours (h). The plates are removed after incubation, and the morphology of the colonies on the solid medium is observed, including shape, color, size, surface, edge, elevation, transparency, etc. The single bacteria with different colony morphology are selected for partition scribing and incubated at 37° C. for 48 h, then the colonies are microscopically examined and photographed and recorded. Then purified colonies are picked up with sterile toothpicks and incubated anaerobically at 37° C. for 48 h. After 48 h of incubation, 1.6 mL of the liquid is dispensed into a glycerol tube, labeled and mixed well, and then stored at −80° C. The genomes of the bacteria are extracted by bacterial genome extraction kit, and 16s rRNA is amplified by 27F/1492R primer and sent to Shanghai Sangon for sequencing, then the sequenced result is compared to NCBI database and it is identified as Bacillus coagulans.

The gene sequence of 16s rRNA is shown in the following SEQ ID No.1:

Tgcggaccttttaaagcttgcttttaaaaggttagcggcggacgggtga
gtaacacgtgggcaacctgcctgtaagatcgggataacgccgggaaacc
ggggctaataccggatagttttttcctccgcatggaggaaaaaggaaag
acggcttcggctgtcacttacagatgggcccgcggcgcattagctagtt
ggtggggtaacggctcaccaaggcaacgatgcgtagccgacctgagagg
gtgatcggccacattgggactgagacacggcccaaactcctacgggagg
cagcagtagggaatcttccgcaatggacgaaagtctgacggagcaacgc
cgcgtgagtgaagaaggccttcgggtcgtaaaactctgttgccggggaa
gaacaagtgccgttcgaacagggcggcgccttgacggtacccggccaga
aagccacggctaactacgtgccagcagccgcggtaatacgtaggtggca
agcgttgtccggaattattgggcgtaaagcgcgcgcaggcggcttctta
agtctgatgtgaaatcttgcggctcaaccgcaagcggtcattggaaact
gggaggcttgagtgcagaagaggagagtggaattccacgtgtagcggtg
aaatgcgtagagatgtggaggaacaccagtggcgaaggcggctctctgg
tctgtaactgacgctgaggcgcgaaagcgtggggagcaaacaggattag
ataccctggtagtccacgccgtaaacgatgagtgctaagtgttagaggg
tttccgccctttagtgctgcagctaacgc.

### Embodiment 2 Application of Bacillus coagulans BC69

### 1. Materials and Methods

### 1.1 Experimental Animals

Forty-six-week-old specific pathogen free (SPF) healthy male Kunming mice, weighing (25±5) g, are purchased from Chongqing Medical University. During the feeding period, the mice in each group are supplied with water freely in the feeding environment as follows: semi-circular lighting during day and night, constant humidity, and temperature controlled at 22-25° C.

### 1.2 Experimental Methods

### 1.2.1 Preparation of Drugs

Preparation of loperamide hydrochloride suspension (1 milligram per milliliter, mg/mL): 50 capsules of loperamide hydrochloride (2 mg/capsule) are used, the drug powder is removed out from the capsules and mixed, then added with sterile water to 100 mL; the suspension is prepared before use.

Activated carbon suspension: gum Arabic of 50 g is added with 400 mL of water and boiled until the solution is transparent; then activated carbon of 25 g is added into the transparent solution and boiled for 3 times; after the solution is cooled, it is added with water and the volume is fixed to 500 mL to obtain the activated carbon suspension (50 g/L), which is stored in the refrigerator at 4° C.

### 1.2.2 Modeling Method

After a week of adaptive culture, 40 mice are randomly divided into 5 groups, with 8 mice in each group, namely the group of normal control (NC), group of model (GM), BC69 low-dose group (BC-L), BC69 high-dose group (BC-H) and bisacodyl drug treatment group (Bis). During the experiment, saline gavage of 0.25 mL is administered daily to NC and GM mice; from day 1 to day 4, all mice except NC mice are additionally gavaged with loperamide hydrochloride suspension (1 mg/mL) 0.25 mL twice daily (at 9:00 a.m. and 2:00 p.m. respectively) to induce a functional constipation model of mice; after gavage on day 4, all mice are fasted from food but not water for 16 h; from day 5 to day 11, all groups are gavaged with Bacillus coagulans BC suspension 0.25 mL every day, where the concentrations are 1.0×10⁸ colony-forming units per milliliter (CFU/mL) and 4.0×10⁹ CFU/mL respectively; mice of group Bis are gavaged with bisacodyl aqueous solution at 100 milligrams per kilogram (mg/kg) per day; after the completion of gavage on day 11, all groups of mice are fasted from food but not water for 16 h.

Animal models play an important role in studying the mechanism of functional constipation, and the selection of modeling animals and methods of modeling vary for different research purposes. Consequently, the establishment of animal models that share common pathophysiological changes with human constipation is of great importance for exploring the pathogenesis of constipation and new therapeutic approaches. One of the main modalities for functional constipation is drug modelling, and loperamide hydrochloride is one of the commonly used drugs. It is a peripheral opioid receptor agonist, with the mechanism of inhibiting intestinal water secretion and colonic peristalsis, delaying fecal evacuation and intestinal luminal transport, and developing constipation symptoms by reducing the number, weight and water content of fecal pellets in animals. In this experiment, loperamide hydrochloride is used to induce functional constipation in mice, and the number of fecal pellets and fecal water content within 5 h after modeling with loperamide hydrochloride are significantly reduced compared with those in the blank control group, indicating that the modeling is successful.

### 1.2.3 Measurement of Intestinal Propulsion Rate

After completion of modeling, mice are fasted overnight to empty their intestinal contents. The intestinal propulsion rate of the mice is measured on the morning of the second day, with a specific method as follows: each mouse is orally instilled with 0.25 mL of activated carbon suspension, and then executed by cervical spinal dislocation after 20 min; the abdominal cavity of the mice is opened rapidly, with the whole intestine from the pylorus to the anus taken out and straightened without tension, then the whole length of the intestine is measured as L1, the travel length of activated carbon in the intestine is measured as L2, and the intestinal propulsion rate of mice is calculated as D=L2/L1×100%.

### 1.2.4 Measurement of Fecal Water Content

During the experiment, the cages are changed to new cages every morning after administration, and before the administration in the afternoon, the feces of mice between the two administrations are collected, and the wet weight of feces is weighed and recorded as W1 to compare the difference in fecal volume between groups; then the feces of mice in each group are put into the blower for high temperature drying and dehydration; the mice feces are weighed again and the dry weight is recorded as W2; the fecal water content of mice is calculated as R=(W1-W2)/W1×100%.

### 1.2.5 Sample Collection and Preservation

Serum: the whole blood is collected by orbital blood sampling, rested for 2 h in a refrigerator at 4° C., then centrifuged at 3,000 revolutions per minute (r/min) for 10 min at 4° C., and the serum is collected from the upper layer; the obtained serum is then divided into appropriate amounts and stored in a refrigerator at −80° C. for later use.

Small intestine: after the mice are executed, the small intestine is dissected from the pylorus down to the ileocecal region; the feces in the small intestine is carefully cleaned out and a 0.5 cm section of the small intestine is fixed in tissue fixative and used as a sample for tissue sectioning, while the remaining small intestine is frozen in liquid nitrogen and stored at −80° C. in the refrigerator as a sample for subsequent experiments.

### 1.2.6 Hematoxylin and Eosin (H&E) Staining

After 24 h of fixation in the tissue solution, the small intestine is stained with H&E and then observed through an orthomosaic microscope for the pathological morphology of the small intestinal tissue of constipated mice.

### 1.2.7 Determination of Gastrointestinal Hormone Levels in Serum

The contents of endothelin (ET), gastrin (Gas), motilin (MTL), substance P (SP), somatostatin (SS) and vasoactive intestinal peptide (VIP) in serum are determined according to the instructions of the enzyme-linked immuno sorbent assay (ELISA) kit.

### 1.2.8 Real-Time Quantitative Fluorescence Polymerase Chain Reaction (RT-qPCR)

Messenger ribonucleic acid (mRNA) expressions of COX-2, c-kit, NFκB, iNOS, eNOS, nNOS and SCF are detected by RT-qPCR, and total RNA is extracted and complementary deoxyribonucleic acid (cDNA) reverse transcription is carried out according to the instructions of RNA extraction kit and cDNA reverse transcription kit; then a microspectrophotometer is used to identify the purity of the RNA at 260/280 nanometers (nm). Actin (actin) is used as a standardized internal reference for RT-qPCR analysis, and final RT-qPCR product expression is determined using the 2^{-ΔΔCt} method.

### 1.2.9 Data Analysis

Data from different groups are expressed as mean±standard deviation (SD), and the significant difference between the two groups is determined by the t-test, and the subsequent temporary analysis test of ANOVA and Dunnett is carried out for comparing that of more than two groups, and GraphPad 7.0 is used for analysis.

### 2. Experimental Results

### 2.1 Effect of Bacillus coagulans BC69 on the Body Weight of Constipated Mice

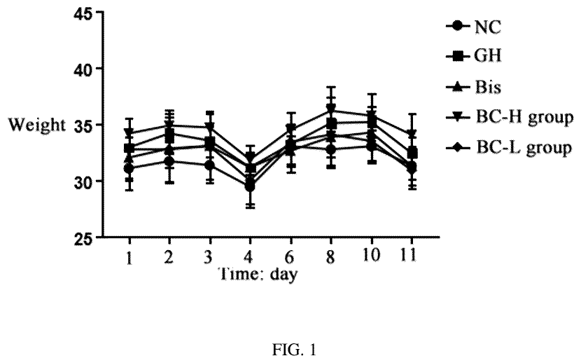

In order to evaluate the weight change of constipated mice throughout the experiment, the weight of mice in each group is measured and recorded, and the results are shown in FIG. 1. The body weight of all groups of mice increases during the modeling period (day 1 to day 4), and the weight increase is more obvious in the induced group compared with that of NC due to the constipation of the induced mice. After the start of treatment, the body weight of mice in each group increases again from the 6^{th} day onwards, among which the body weight of GM mice increases significantly, while the body weight of mice gavaged with Bacillus coagulans BC69 tends to decrease at the later stage of the experiment, but the analysis shows that there is no significant difference between the body weight of mice in each group (p>0.05). In this way, it is suggested that loperamide hydrochloride induced constipation in mice causes a certain degree of weight increase, while gavage of Bacillus coagulans BC69 can better alleviate the weight increase in constipated mice.

### 2.2 Effect of Bacillus coagulans BC69 on Fecal Water Loss Rate in Constipated Mice

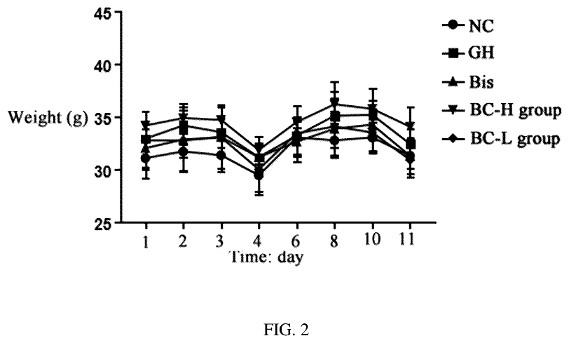

The fecal water content is an important index to evaluate the success of constipation model. In patients with constipation, the intestinal motility is weakened and the feces are dry, resulting in a decrease in the fecal water content. As shown in FIG. 2, during the modeling period (day 1 to day 4), the water contents in the feces of all groups of mice, except for the group of normal control, are decreased to different degrees, and by the last day of modeling, the fecal water contents of the model group are significantly lower than that of the group of normal control (p<0.05), indicating the modeling is successful. After starting the intervention treatment from day the fecal water contents of mice gavaged with Bacillus coagulans BC69 is increased compared with that of GM mice, and the fecal water content of mice in BC-H is close to that of mice in NC and Bis, indicating that Bacillus coagulans BC69 can alleviate the symptoms of constipation by increasing the fecal water content of mice.

### 2.3 Effect of Bacillus coagulans BC69 on Small Intestine Propulsion Rate in Constipated Mice

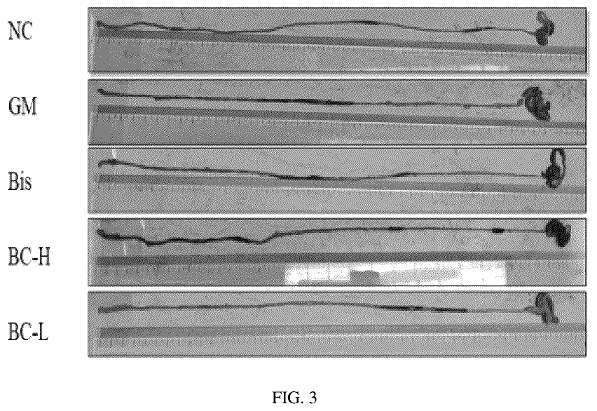

As can be seen from FIG. 3, there is a shortening of the small intestine in GM mice compared with that of the rest of the groups, but the difference is not significant, and there is no significant difference between the length of the small intestine in the mice of remaining groups (p>0.05), indicating that constipation affects the length of the small intestine to some extent, but the results of the effect are not significant. As shown in Table 1, the small intestinal propulsion rate of GM mice is 53.7%, significantly lower than that of NC (p<0.05). In contrast, the small intestinal propulsion rate of constipated mice gavaged with Bacillus coagulans BC69 is significantly improved compared with that of GM mice (p<0.05), with the administration effect of BC-H being more significant. The results suggest that Bacillus coagulans BC69 promotes small intestinal peristalsis and accelerates the propulsion of activated carbon in the small intestine, thus providing certain improvement in constipation.

TABLE 1
Small intestinal propulsion rate

| Groups | Propulsion rate % |
|---|---|
| NC | 0.897 ± 0.04 |
| GM | 0.537 ± 0.08 |
| Bis | 0.761 ± 0.11 |
| BC-H | 0.878 ± 0.03 |
| BC-L | 0.867 ± 0.06 |

Note: NC: group of normal control; GM: constipation model group; Bis: bisacodyl drug treatment group; BC-H: BC high-dose group; BC-L: BC low-dose group.

### 2.4 Effect of Bacillus coagulans BC69 on Intestinal Histomorphology in Constipated Mice

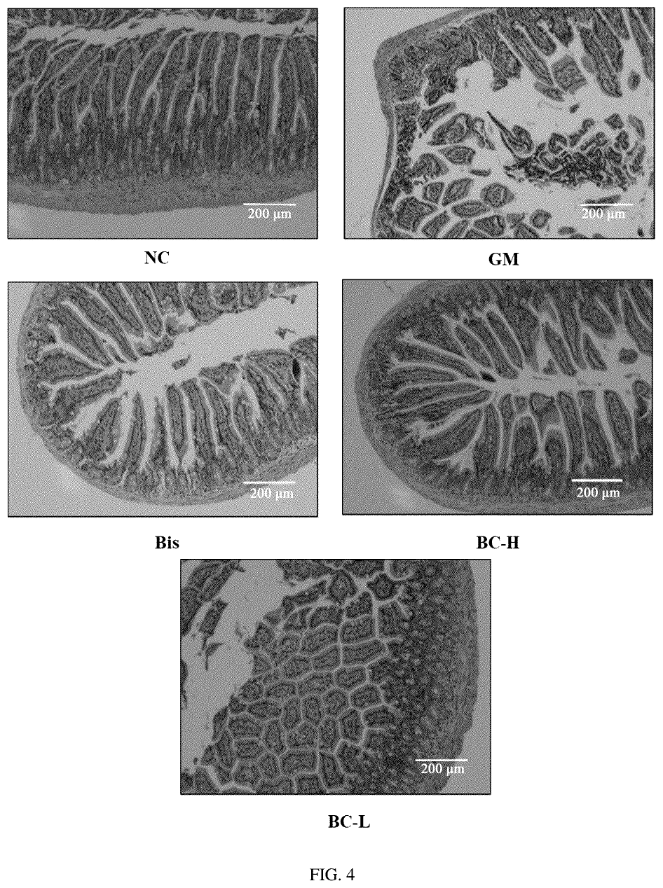

Intestinal peristaltic function is affected to varying degrees when the small intestinal villi are damaged, whereas slowed intestinal peristalsis is one of the factors causing constipation, so the integrity of the small intestinal villi is equally important in the evaluation of constipation. As shown in FIG. 4, the small intestinal villi of NC mice are neatly and uniformly arranged without fractures or wrinkles, and the goblet cells are abundant; the small intestinal villi of GM mice show severe fractures and wrinkles, the brush border appears in disorganization, and the goblet cells are incomplete. Although the small intestinal villi of BC-L and BC-H also show some degree of wrinkling and fracture, the small intestinal villi are obviously more complete than those of GM mice, and the small intestinal villi of BC-H mice is more complete.

### 2.5 Effect of Bacillus coagulans BC69 on Gastrointestinal Hormones in Serum of Constipated Mice

Gastrointestinal hormones exert important regulatory effects on absorption, motility, secretion and immunity of the digestive system through different signals, and are also closely related to the occurrence of constipation. According to the results shown in Table 2, the level of substance P (SP) is significantly lower in GM serum compared to that of NC (p<0.05), and the levels of ET, Gas, MTL, SS, and VIP are up-regulated. The SP levels of BC-L and BC-H mice are significantly increased compared to those of GM mice (p<0.05), and the increase in SP concentration is more pronounced with the increase of the gavage concentration of Bacillus coagulans BC69. Additionally, the levels of ET-1, Gas, MTL, SS, and VIP are downregulated, also showing a more pronounced downregulation effect as the gavage concentration of Bacillus coagulans BC increases. The detection results of serum in the treatment group suggest that gavage of Bacillus coagulans BC69 to constipated mice relieves constipation by regulating the secretion of gastrointestinal hormones, thereby accelerating gastrointestinal transport.

TABLE 2
Serum indicators

| | ET-1 | Gas | MTL | SP | SS | VIP |
|---|---|---|---|---|---|---|
| | (pg/mL) | (pg/mL) | (pg/mL) | (pg/mL) | (pg/mL) | (pg/mL) |
| NC | 20.69 ± 0.92 | 8.80 ± 0.84 | 54.86 ± 2.55 | 54.93 ± 2.39 | 26.88 ± 0.85 | 34.21 ± 1.30 |
| GM | 24.07 ± 1.64 | 11.82 ± 1.10 | 62.93 ± 3.65 | 49.44 ± 3.93 | 30.06 ± 1.65 | 37.91 ± 2.15 |
| Bis | 21.44 ± 1.28 | 10.09 ± 0.69 | 57.89 ± 2.65 | 51.60 ± 3.48 | 28.09 ± 1.89 | 36.13 ± 2.37 |
| BC-H | 23.80 ± 1.85 | 7.99 ± 1.09 | 56.05 ± 4.79 | 57.32 ± 6.38 | 27.52 ± 0.85 | 33.971 ± 3.93 |
| BC-L | 23.81 ± 1.99 | 8.71 ± 0.96 | 63.32 ± 5.58 | 57.91 ± 3.28 | 28.46 ± 1.66 | 35.19 ± 2.47 |

Note:
NC: group of normal control; GM: constipation model group; Bis: bisacodyl drug treatment group; BC-H: BC high-dose group; BC-L: BC low-dose group; ET-1: endothelin-1; Gas: gastrin; MTL: motilin; SS: somatostatin; VIP: vasoactive intestinal peptide.

### 2.6 Effect of Bacillus coagulans BC69 on the Expression Level of Related mRNA in Small Intestine of Constipated Mice

The mRNA expressions of COX-2, c-kit, NFκB, iNOS, eNOS, nNOS and SCF in small intestine of constipated mice are analyzed by RT-qPCR. From the results as shown in Table 3, the expressions of COX-2, NFκB and SCF in small intestine of GM mice are up-regulated, while the expressions of c-kit, iNOS, eNOS and nNOS are down-regulated. The expressions of COX-2, NFκB, and SCF in small intestinal tissues are slightly regulated and the expressions of c-kit, iNOS, eNOS, and nNOS are up-regulated in the treatment group of Bacillus coagulans BC compared to GM, and the expression effect improves significantly with increasing concentration of the bacterial solution. The mRNA expression results indicate that gavage of Bacillus coagulans BC69 to constipated mice affects gene expression in mouse small intestinal tissues.

TABLE 3
mRNA expression levels

| | COX-2 | c-kit | NFκB | iNos | eNos | nNos | SCF |
|---|---|---|---|---|---|---|---|
| NC | 1.00 ± 0.07 | 1.00 ± 0.07 | 1.08 ± 0.50 | 1.01 ± 0.11 | 1.00 ± 0.09 | 1.07 ± 0.20 | 1.02 ± 0.24 |
| GM | 5.06 ± 0.35 | 0.66 ± 0.06 | 2.85 ± 2.42 | 0.64 ± 0.18 | 0.26 ± 0.02 | 0.40 ± 0.09 | 1.70 ± 0.32 |
| Bis | 0.43 ± 0.05 | 0.08 ± 0.01 | 2.21 ± 0.84 | 0.04 ± 0.00 | 0.03 ± 0.00 | 0.04 ± 0.00 | 3.66 ± 0.62 |
| BC-H | 0.41 ± 0.02 | 0.12 ± 0.02 | 1.80 ± 0.20 | 0.16 ± 0.07 | 0.24 ± 0.04 | 0.10 ± 0.01 | 11.63 ± 1.83 |
| BC-L | 1.42 ± 0.31 | 0.57 ± 0.12 | 1.47 ± 1.93 | 0.47 ± 0.08 | 0.21 ± 0.06 | 0.05 ± 0.01 | 6.09 ± 2.66 |

Note:
NC: group of normal control; GM: constipation model group; Bis: bisacodyl drug treatment group; BC-H: BC high-dose group; BC-L: BC low-dose group.

### 3. Conclusion

Constipation is associated with dysbiosis of the intestinal microbiota, and reduced abundance of intestinal flora may be a potential cause or consequence of impaired intestinal motility. In the present application, the effect of Bacillus coagulans BC69 on constipation amelioration and laxative effect in constipated model mice is investigated. After gavage with different doses of Bacillus coagulans BC69, the functional constipation of mice is significantly improved, with the most significant effect of BC69 solution of order of magnitude 4.0×10⁹ CFU/mL. High dose of Bacillus coagulans BC69 solution is found to significantly promote the small intestinal propulsion of mice, while increasing the number and quality of 5-h black stools, indicating a good laxative effect.

Enteric nerve parameters secreted by the enteric nerve network in the gastrointestinal tract act as neuromodulators and neurotransmitters to facilitate intestinal motility and transport of contents. Gastrointestinal hormones are a group of small active peptides produced by endocrine cells and nerve cells in the gastrointestinal tract. Some gastrointestinal hormones are also present in the central nervous system, which is why they are also referred to as cerebrointestinal peptides or neurotransmitters. After binding to receptors on target cells, gastrointestinal hormones possess important regulatory effects on absorption, movement, secretion and immunity of the digestive system by means of different signals, and are also closely related to the occurrence of constipation. In the present application, the treatment of Bacillus coagulans BC69 significantly improves the levels of SP in serum and inhibits the levels of ET-1, SS and VIP in serum, and the effect is concentration-dependent, and the effects of Bacillus coagulans BC69 solution at high doses on the regulation of gastrointestinal hormones in mouse serum are more pronounced.

In addition to directly regulating these neurotransmitters, Bacillus coagulans BC69 also affects mRNA expression in the small intestine of constipated mice. Interstitial cells of Cajal (ICC) are important in intestinal motility, and some studies have shown that ICC numbers are decreased in the colon of patients with chronic constipation, which has been suggested to be caused by downregulation of c-Kit expression. The c-Kit receptor (CD117), also known as stem cell factor receptor, is a transmembrane protein with tyrosine kinase activity and is one of the specific markers of ICC. A natural ligand for c-Kit is SCF, and the SCF/c-Kit signaling pathway is critical for ICC proliferation, differentiation and phenotype maintenance. It is demonstrated by the results of the present application that the mRNA levels of c-Kit are lower in model mice and the ratio of SCF/c-Kit is significantly higher after Bacillus coagulans BC69 treatment compared to that of constipated mice. Moreover, the expressions of inflammatory factors COX-2, NFκB, iNOS, eNOS, and nNOS are depressed in the colonic tissues of Bacillus coagulans BC69-treated mice.

The above embodiments describe only the preferred methods of the present application, and do not limit the scope of the present application. Without departing from the spirit of the design of the present application, all kinds of deformations and improvements made to the technical schemes of the present application by a person of ordinary skill in the art shall fall within the scope of protection determined by the claims of the present application.

What is claimed is:

1. An application of a Bacillus coagulans in preparing products for preventing and treating constipation, wherein the Bacillus coagulans is preserved in China General Microbiological Culture Collection Center on Nov. 10, 2021, with a preservation number of CGMCC No. 23766 and a preservation address of the Institute of Microbiology, Chinese Academy of Sciences, No. 3, Yard No. 1 Beichen West Road, Chaoyang District, Beijing; and
the constipation refers to constipation caused by loperamide hydrochloride.

2. An application of a Bacillus coagulans in preparing products for preventing or treating constipation, wherein the Bacillus coagulans is preserved in China General Microbiological Culture Collection Center on Nov. 10, 2021, with a preservation number of CGMCC No. 23766 and a preservation address of the Institute of Microbiology, Chinese Academy of Sciences, No. 3, Yard No. 1 Beichen West Road, Chaoyang District, Beijing; and
the constipation refers to constipation caused by loperamide hydrochloride.


SEQUENCE LISTING (1 sequence)

SEQ ID NO: 1 - DNA, 813 bp, synthetic construct
  source 1..813: mol_type = other DNA
tgcggaccttttaaagcttgcttttaaaaggttagcggcggacgggtgagtaacacgtgggcaacctgcctgtaagatcgggataacgccgggaaaccggggctaataccggatagttttttcctccgcatggaggaaaaaggaaagacggcttcggctgtcacttacagatgggcccgcggcgcattagctagttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgc